(19) United States
(12) Patent Application Publication
(10) Pub. No.: US 2008/0027312 A1
(43) Pub. Date: Jan. 31, 2008

(54) DISPLAYING OBJECT ORIENTATIONS ON BALL JOINTS

(76) Inventor: Robert Dick, Munich (DE)

Correspondence Address: RENNER, OTTO, BOISSELLE & SKLAR, LLP, Nineteenth Floor, 1620 Euclid Avenue, Cleveland, OH, 44115-2191, US

(21) Appl. No.: 11/778,680

(22) Filed: Jul. 17, 2007

Related U.S. Application Data
(60) Provisional application No. 60/821,302, filed on Aug. 3, 2006.

(30) Foreign Application Priority Data
Jul. 25, 2006 (EP) 06015447

(51) Int. Cl. A61B 1/00; A61B 5/05

(52) U.S. Cl. 600/424; 606/102

(57) ABSTRACT

A method for displaying, in the field of medical technology, an orientation of an object on a ball joint, includes: defining a zero angle projecting out of a joint cavity on the ball joint, wherein directions are defined as anatomical directions; defining a preferred direction of the object, wherein the preferred direction is to be oriented in relation to the zero angle; and graphically displaying the object orientation, wherein a deviation between the preferred direction and the zero angle is displayed as a magnitude, and the relative orientation between the preferred direction and at least one anatomical direction is displayed as a circumferential representation having a proportional value corresponding to the at least one anatomical direction.

### RELATED APPLICATION DATA

This application claims priority of U.S. Provisional Application No. 60/821,302 filed on Aug. 3, 2006, which is incorporated herein by reference in its entirety.

### FIELD OF THE INVENTION

The invention relates to the display of ball joints in the context of surgical procedures and, more particularly, to a device and method for displaying the orientation of an object on a ball joint.

### BACKGROUND OF THE INVENTION

The placement of medical ball joint implants, in particular the insertion of cavity implants for hip joints, can be assisted by medical navigation. Such medical navigation enables the position of the implant and/or the patient (and thus their relative positions) to be determined and tracked. This information can be provided to the physician performing the treatment via visual assistance (e.g., via a screen output).

As an aid for the surgeon, the position of a hip implant can be described by two angles that are output within the context of navigation data. The two angles can be the inclination and anteversion, and the deviation from one position to another may be described by a change in anteversion and a change in inclination.

A typical definition of anteversion is the angular movement when raising the leg forward direction, while movement in the opposite direction (e.g., when retracting the leg) is referred to as retroversion. Inclination and declination are movement directions opposite to each other and perpendicular to anteversion. The same definitions apply to the movement of the upper arm in the shoulder ball joint, for example.

A problem with the above terms is that the medical literature describes different ways of calculating inclination and anteversion angles. This can lead to misunderstandings with respect to positioning an implant, which are not tolerable in medical surgery. Additionally, outputting two angles, i.e., the angles for each of anteversion and inclination, is not particularly clear, which can make it difficult to correct for position errors, particularly if the physician performing the treatment does not have extensive experience.

### SUMMARY OF THE INVENTION

The present invention optimizes the display of object orientations on ball joints used in the medical field. More specifically, an unmistakable representation of the object and/or ball joint can be provided that allows the position of the object to be simply and clearly rendered and corrected.

A method for displaying the orientation of an object on a ball joint in the field of medical technology includes:
a zero angle projecting out of the joint cavity on the ball joint is defined, and directions are defined as anatomical directions;
the object exhibits a defined preferred direction which is to be orientated in relation to the zero angle; and wherein
the object orientation is graphically displayed, wherein a deviation between a preferred direction and the zero angle is displayed in terms of magnitude, e.g., numerically, and the orientation relationship between the preferred direction and the anatomical directions is displayed on a circumferential representation as a proportional value of the applicable anatomical directions.

The surgeon requires clear and comprehensible information on how the position of the implant has changed, for example, with respect to his original planning. The method described herein can separate this required information into an angular deviation (preferred direction/zero angle) and directional information (orientation relationship between the preferred direction and the anatomical directions). Since the angular information is displayed independent of the directional information, it is unambiguous and unmistakable. Further, displaying the directional information as a circumferential representation with proportional values of anatomical directions makes the corresponding deviation easily visible and comprehensible. When viewing the representation, the surgeon knows exactly how large the deviation is (e.g., as a magnitude in degrees) and in which direction it extends. Using such a visual assistance, a deviation can be simply corrected by a corresponding angular adjustment in exactly the opposite direction.

In a preferred embodiment, the anatomical directions are also angular directions, which lends itself to ball joints on the human body.

The preferred direction of the object can be defined by an axis or plane of the object. It is also possible, however, to define the preferred direction of the object using an axis or plane of an object attached to the object, for example a holder for the object.

The object can be an implant, in particular a joint cavity implant, and the preferred direction can be the axial direction of the implant holding and placing device.

The anatomical directions described above, as appropriate, can be any known and typical anatomical directions. An exemplary selection is given below:
anteversion or retroversion;
inclination or declination;
abduction or adduction;
eversion or inversion;
rotation as an angular deviation from the anatomical normal position, in particular from the zero angle;
elevation;
transversal, sagittal, longitudinal, frontal, medial, lateral.

A method can be configured such that the circumferential representation is an image of a closed circumference, in particular a circular or elliptical circumference (polygonal circumferences are also possible). The closed circumference can be sub-divided by two perpendicularly superimposed anatomical displays, in particular main direction displays. A pointer means can display the proportional value of the current anatomical angular direction for the object. The pointer means can include all possible shapes, e.g., that of an actual pointer/pointing arrow or also the shape of bars (also colored bars) that are separated by a delineation, the position of which can change depending on the position of the object.

Also provided herein is a program which, when it is running on a computer or is loaded onto a computer, causes the computer to perform a method such as described herein, and to a computer program storage medium comprising such a program.

Further, a medical displaying device for displaying the orientation of an object on a ball joint includes:
a data storing and processing means that stores anatomical data, by means of which a zero angle projecting out of the joint cavity on the ball joint is defined, and directions are defined which are defined as anatomical directions, and by means of which a preferred direction of the object is defined which is to be orientated in relation to the zero angle; and
a graphical processing unit and a graphical output by means of which the object orientation is graphically displayed, wherein the deviation between the preferred direction and the zero angle is displayed numerically, and the orientation relationship between the preferred direction and the anatomical directions is displayed on a circumferential representation as a proportional value of the applicable anatomical directions.

### BRIEF DESCRIPTION OF THE DRAWINGS

The forgoing and other features of the invention are hereinafter discussed with reference to the drawing.

FIG. 1 illustrates a top view onto an exemplary joint cavity with an exemplary representation of the anatomical directions.

FIG. 2 illustrates an exemplary orientation deviation for a hip joint implant in accordance with the invention.

FIG. 3 illustrates the hip joint of FIG. 2 with a different orientation deviation.

FIG. 4 is a block diagram of an exemplary computer system that can be used to carry out the method in accordance with invention.

### DETAILED DESCRIPTION

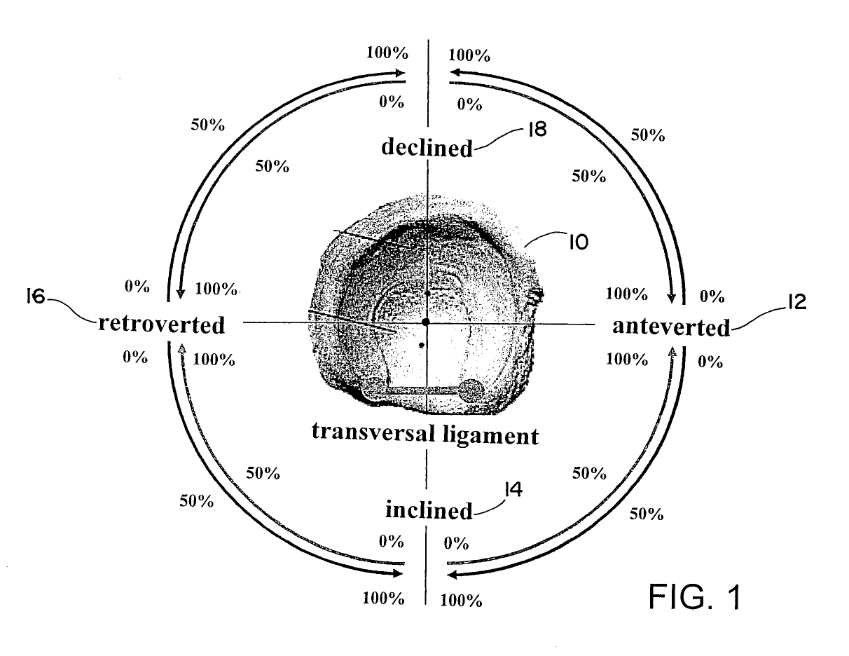

FIG. 1 illustrates an exemplary hip joint cavity 10 that is localized and/or tracked (positionally detected) using, for example, a medical navigation and/or tracking system. Also shown in FIG. 1 are anatomical directions that are used in conjunction with the method described herein. The two main directions shown in FIG. 1 are the anteversion 12 and the inclination 14, wherein the opposite direction of the anteversion is the retroversion 16 and the opposite direction of the inclination is the declination 18. These two directions are perpendicularly superimposed, and in the circumferential representation (in which proportional values from 0 to 100 percent are given in each of the quadrants) a particular value for anteversion, retroversion, inclination and declination can be assigned to each point on the circumference. The directional information can be displayed along the anatomical directions shown, in the origin plane or perpendicular to the origin plane.

In the example of FIG. 1, the transversal ligament is used to define the main directions of anteversion and inclination, wherein the anteversion lies parallel to the transversal ligament and the inclination lies perpendicular to it. Other definitions of anteversion and inclination could of course also be chosen.

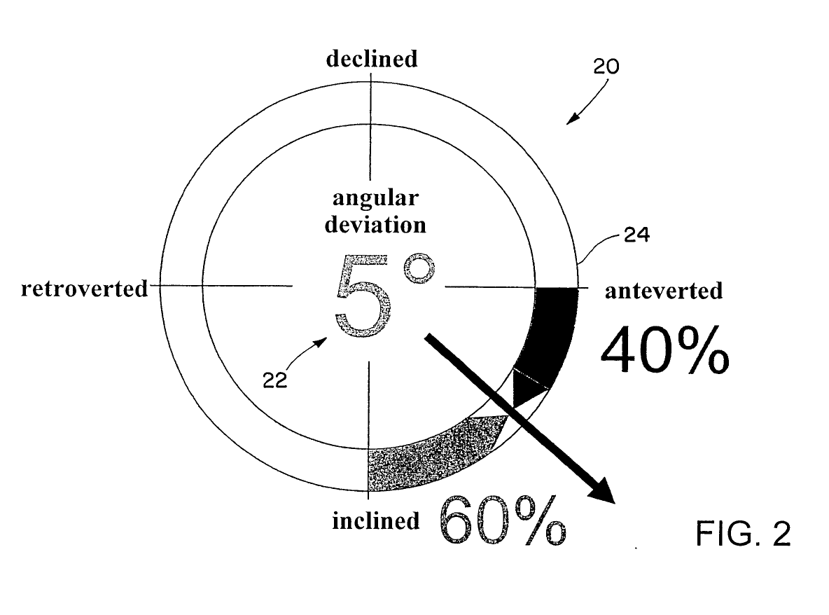

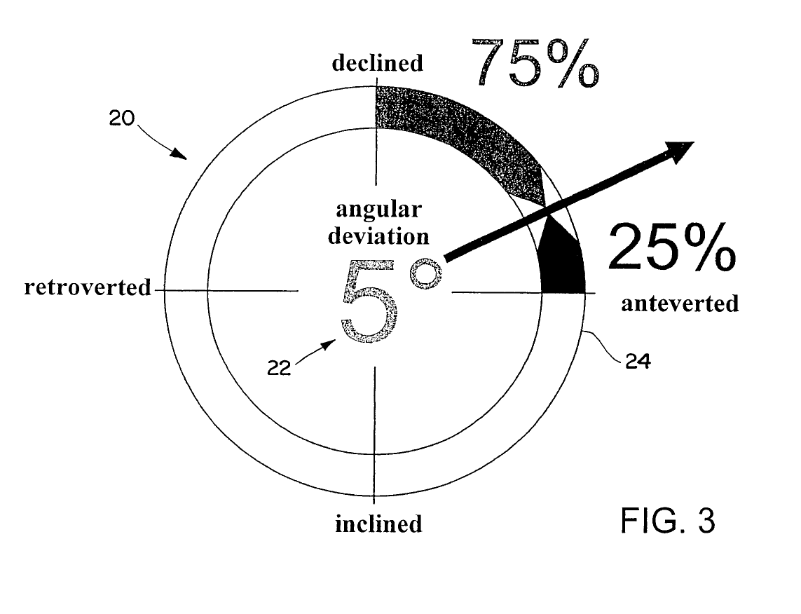

Instead of the typical medical information relating to just two angular deviations relative to ambiguously calculated angular directions, e.g., merely with deviations in inclination and anteversion, the deviation information of FIG. 1 is split into two items of information, i.e., an absolute angular deviation and directional information. The information split in this way also can be separately displayed, wherein the angular deviation may be directly ascertained, either from one plane to another or from one vector to another. The displaying principle is discussed with respect to FIGS. 2 and 3.

The orientation deviation of the object, as can be seen in the example shown in FIG. 2, can be displayed by a circular circumferential representation 20. In the center of the exemplary representation is an absolute value for the angular deviation 22. The representation can be envisaged as if the joint cavity were also shown, as in FIG. 1. The angular deviation of 5 degrees displayed separately in the center then shows that the object (e.g., a placing or mounting device for the implant to be inserted, on which the implant is held) deviates from the planned direction by 5 degrees. The information on the direction in which the deviation extends is provided by the circumferential representation, e.g., the circular bar 24, and in the case of FIG. 2, the direction is 60 percent inclined and 40 percent anteverted. The surgeon, based on the display, then knows that he has to move his implant carrier (placing device) in exactly the opposite direction in order to correct the deviation in inclination and anteversion, and only by a small angle of 5 degrees. If he moves the implant carrier, which is also navigated and/or tracked, the graphical display also will adapt, until the angular deviation is given as 0 percent and a pointer can no longer be seen. The same applies to a deviation such as is for example shown in FIG. 3, in which there is a declination of 75 percent and an anteversion of 25 percent.

Another example may be a deviation of 100 percent in the anteversion direction, which would result in an arrow that points horizontally to the right-hand side with a display of 100 percent anteverted. Compared to a compass, this would be a direction extending exactly to the “east”.

It would in principle also be conceivable to make the arrow longer or shorter depending on the size of the angular deviation, such that separate zero angle deviation information is thus provided, in particular as additional information.

By separately rendering the angular deviation and directional deviation, the deviations can be quickly and specifically corrected, even by surgeons who do not yet have very extensive experience in the corresponding medical field.

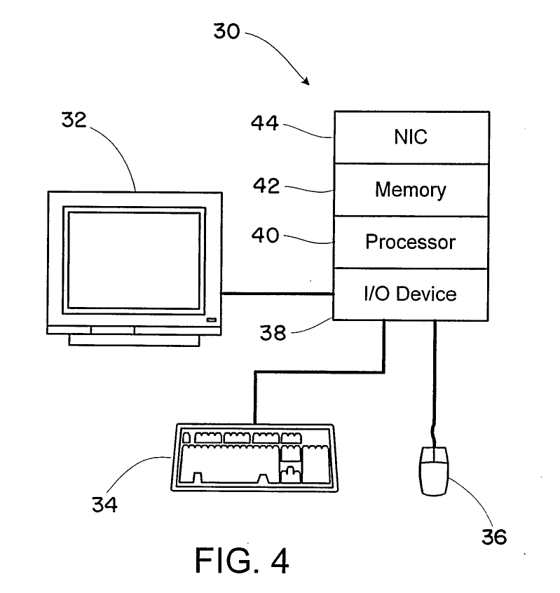

Moving now to FIG. 4 there is shown a block diagram of an exemplary computer system 30 that may be used to implement one or more of the methods described herein. The computer system may be a stand alone system, or it may be part of the navigation system described herein. The computer system 30 may include a display 32 for viewing system information, and a keyboard 34 and pointing device 36 for data entry, screen navigation, etc. A computer mouse or other device that points to or otherwise identifies a location, action, etc., e.g., by a point and click method or some other method, are examples of a pointing device 36. Alternatively, a touch screen (not shown) may be used in place of the keyboard 34 and pointing device 36. The display 32, keyboard 34 and mouse 36 communicate with a processor via one or more input/output devices 38, such as a video card and/or serial port (e.g., a USB port or the like). As will be appreciated, the video card can include a dedicated graphical processing unit (GPU) for generating graphics for the display.

A processor 40, such as an AMD Athlon 64® processor or an Intel Pentium IV® processor, combined with a memory 42 execute programs to perform various functions, such as data entry, numerical calculations, screen display, system setup, etc. The memory 42 may comprise several devices, including volatile and non-volatile memory components. Accordingly, the memory 42 may include, for example, random access memory (RAM), read-only memory (ROM), hard disks, floppy disks, optical disks (e.g., CDs and DVDs), tapes, flash devices and/or other memory components, plus associated drives, players and/or readers for the memory devices. The processor 40 and the memory 42 are coupled using a local interface (not shown). The local interface may be, for example, a data bus with accompanying control bus, a network, or other subsystem.

The memory may form part of a storage medium for storing information, such as application data, screen information, programs, etc., part of which may be in the form of a database. The storage medium may be a hard drive, for example, or any other storage means that can retain data, including other magnetic and/or optical storage devices. A network interface card (NIC) 44 allows the computer system 30 to communicate with other devices.

A person having ordinary skill in the art of computer programming and applications of programming for computer systems would be able in view of the description provided herein to program a computer system 30 to operate and to carry out the functions described herein. Accordingly, details as to the specific programming code have been omitted for the sake of brevity. Also, while software in the memory 42 or in some other memory of the computer and/or server may be used to allow the system to carry out the functions and features described herein in accordance with the preferred embodiment of the invention, such functions and features also could be carried out via dedicated hardware, firmware, software, or combinations thereof, without departing from the scope of the invention.

Computer program elements of the invention may be embodied in hardware and/or in software (including firmware, resident software, micro-code, etc.). The invention may take the form of a computer program product, which can be embodied by a computer-usable or computer-readable storage medium having computer-usable or computer-readable program instructions, “code” or a “computer program” embodied in the medium for use by or in connection with the instruction execution system. In the context of this document, a computer-usable or computer-readable medium may be any medium that can contain, store, communicate, propagate, or transport the program for use by or in connection with the instruction execution system, apparatus, or device. The computer-usable or computer-readable medium may be, for example but not limited to, an electronic, magnetic, optical, electromagnetic, infrared, or semiconductor system, apparatus, device, or propagation medium such as the Internet. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner. The computer program product and any software and hardware described herein form the various means for carrying out the functions of the invention in the example embodiments.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A method for displaying, in the field of medical technology, an orientation of an object on a ball joint, comprising:
defining a zero angle projecting out of a joint cavity on the ball joint;
defining a preferred direction of the object, wherein the preferred direction is to be oriented in relation to the zero angle, and wherein directions are defined as anatomical directions; and
graphically displaying the object orientation, wherein a deviation between the preferred direction and the zero angle is displayed as a magnitude, and the relative orientation between the preferred direction and at least one anatomical direction is displayed as a circumferential representation having a proportional value corresponding to the at least one anatomical direction.

2. The method according to claim 1, wherein displaying the magnitude includes numerically displaying the magnitude.

3. The method according to claim 1, wherein defining directions as anatomical directions includes using angular directions as the anatomical directions.

4. The method according to claim 1, wherein defining the preferred direction of the object includes defining an axis or plane of the object or an axis or plane of an object holder attached or couplable to the object.

5. The method according to claim 1, wherein the object is an implant, and the preferred direction is an axial direction of an implant mounting device.

6. The method according to claim 5, wherein the implant is a joint cavity.

7. The method according to claim 1, wherein defining directions as anatomical directions includes using one or more of the following as the anatomical directions:
anteversion or retroversion;
inclination or declination;
abduction or adduction;
eversion or inversion;
rotation as an angular deviation from an anatomical normal position;
elevation; or
transversal, sagittal, longitudinal, frontal, medial, lateral.

8. The method according to claim 1, wherein displaying the relative orientation between the preferred direction and the at least one anatomical direction as a circumferential representation includes displaying an image of a closed circumference that is sub-divided by two perpendicularly superimposed anatomical directional displays, wherein a pointer displays the proportional value of a current angular direction for the object.

9. The method according to claim 8, wherein displaying the image of a closed circumference includes displaying a circular or elliptical circumference.

10. The method according to claim 1, wherein defining the zero angle includes defining the zero angle based on anatomical features of the joint cavity and/or anatomical features surrounding the joint cavity.

11. The method according to claim 10, wherein defining the zero angle based on anatomical features surrounding the joint cavity includes defining the zero angle based on a position of the transversal ligament.

12. A computer program embodied on a machine readable medium for displaying, in the field of medical technology, an orientation of an object on a ball joint, comprising:
code that defines a zero angle projecting out of a joint cavity on the ball joint;
code that defines a preferred direction of the object, wherein the preferred direction is to be oriented in relation to the zero angle, wherein directions are defined as anatomical directions; and
code that graphically displays the object orientation, wherein a deviation between the preferred direction and the zero angle is displayed as a magnitude, and the relative orientation between the preferred direction and at least one anatomical direction is displayed as a circumferential representation having a proportional value corresponding to the at least one anatomical direction.

13. A medical displaying device for displaying the orientation of an object on a ball joint, comprising:
a data storage device operative to store anatomical data;
a processing device operatively coupled to said storage device, said processing device operative to
define a zero angle projecting out of a joint cavity on the ball joint based on the stored anatomical data, and to define directions as anatomical directions, and
define a preferred direction of the object to be orientated in relation to the zero angle;
a graphical processing unit operatively coupled to said processing device; and
a graphical output device operatively coupled to said graphical processing unit, wherein said graphical processing unit and/or graphical output device are operative to provide a graphical display of the object orientation, and wherein
a deviation between the preferred direction and the zero angle is displayed numerically, and
the relative orientation between the preferred direction and at least one anatomical direction is displayed as a circumferential representation having a proportional value corresponding to the at least one anatomical direction.
